(19) United States
(12) Patent Application Publication
(10) Pub. No.: US 2002/0165392 A1
(43) Pub. Date: Nov. 7, 2002

(54) Method of preparing alkaloids using supercritical fluids from plants

(76) Inventors: Jin Woong Kim, Seoul (KR); Young Hae Choi, Seoul (KR); Ki Pung Yoo, Seoul (KR); Min Jeong Noh, Daejon (KR); Joo Hee Han, Daejon (KR)

Correspondence Address: MONICA MCCORMICK GRAHAM, PH.D., Fish & Richardson P.C., P.A., Suite 3300, 60 South Sixth Street, Minneapolis, MN, 55402, US

(21) Appl. No.: 09/792,306

(22) Filed: Feb. 23, 2001

(30) Foreign Application Priority Data
Feb. 25, 2000 (KR) 2000-9257

(51) Int. Cl. C07C 211/00

(52) U.S. Cl. 546/91; 564/381

(57) ABSTRACT

The invention relates to a method for extracting and preparing alkaloids from plants using supercritical fluid extracting technology and alkaloids prepared by said method. Specifically, the invention relates to a method of preparing alkaloids by mixing plants with carbon dioxide as a major solvent and 1 to 20 parts by weight of at least one alkaline cosolvent selected from a group consisting of methanol, ethanol, water or a mixture thereof wherein 2 to 18 (v/v) % of diethylamine or triethylamine is dissolved in the cosolvent based on 100 parts by weight of carbon dioxide at the temperature of 70 to 90° C. and under 4000 to 6000 PSI of pressure in a supercritical extracting apparatus to extract the alkaloid, and then separating, filtering and purifying the alkaloid using the chromatography, and to alkaloids prepared by said method.

### FIELD OF THE INVENTION

The present invention relates to methods for extracting and preparing alkaloids from plants using supercritical fluid extraction technology, and alkaloids prepared by said method. More specifically, the invention relates to a method for preparing alkaloids by mixing plant material with carbon dioxide o r a mixture of carbon dioxide with at least one cosolvent in a supercritical state at high temperature and pressure to extract the alkaloid from the plant, and then separating and purifying the alkaloids.

### BACKGROUND OF THE INVENTION

Supercritical fluid extraction technology uses fluids above a critical temperature and pressure, and it has been noted as a new environmentally friendly and clean technology that can substitute for the prior art in the fields of the extraction and purification of medicines, foods and petrochemicals. In particular, owing to the rise of the cost of energy resources, environmental damage caused by traditional separation processes and expansion of the demand for new material having special purposes which can not be prepared by gas or liquid processes, intensive research has been conducted in many countries for the past 30 years in order to develop a new process of fluid technology. This new process uses a supercritical fluid extraction technology, whereas the traditional process uses gas and liquid. As a result, the technology using supercritical fluid is rapidly going to affect relevant industry, including fine chemistry, energy, environmental and new materials, and this technology is going to substitute various traditional separation technologies.

Among the various supercritical fluid candidates used, carbon dioxide offers particular advantages. Carbon dioxide is a material that can be produced in the petrochemical and iron manufacturing industry, and an infinite amount exists in nature. Also, carbon dioxide is colorless, odorless and non-toxic to humans and is chemically stable.

In addition to the above, carbon dioxide exhibits a critical temperature (31.1° C.) and pressure (7.4 MPa), lower than any fluids, and, thus, it can easily be adjusted supercritical conditions so that it has great advantages in terms of its efficient energy consumption and environmentally friendly properties. Furthermore, if said technology is employed in the fields for separating and purifying physiologically active natural materials, it can be solved or complemented for the many portions of the problems in which would be produced in the prior process. For example, the problems include human toxicity due to organic solvents in the final products, high costs, the environmental pollution caused by the wasted solvents, denaturalization of the desired components and low extraction selectivity, etc.

Early on, the fields that primarily employed a supercritical fluid extraction technology were limited to non-polar, inexpensive foods and flavors as well as perfume, fatty acids, lipids, steroids and the like. However, the development of various additional technologies relating to the above technology has recently been applied to the extraction and purification process of polar, small and expensive natural medicine.

Due to their increased concern toward the environment, industry has attempted new processes for extracting and purifying natural components to solve the problems of the prior art, which used organic solvents. The problems include, for example, toxicity toward the environment and humans, non-selectivity for the desired component and high costs.

The term “supercritical fluid” refers to a fluid that is above its critical pressure and above its critical temperature. A supercritical fluid has both the gaseous property of being able to penetrate anything and the liquid property of being able to dissolve materials into their components.

All pure materials show gas, liquid and solid phases, depending on the temperature and pressure. When the phase transition curve is observed, it can be seen that an increase in temperature causes an increase in pressure, as a result of an approaching new equilibrium, an increase of vapor pressure is caused, and the difference of the density between the liquid phase and the gas phase is eventually reduced. The difference becomes the same at the critical point. Thus, they cannot be differentiated. Beyond the critical point, liquefaction does not occur even with increased pressure, and vaporization does not occur even with increased temperature.

A supercritical fluid is defined as that having an intermediate property between gas and liquid. Because supercritical fluid demonstrates a great change in the density even minute changes in temperature and pressure, the solubility can easily be adjusted. It also has a property that is uniquely different from gas and liquid.

Namely, it exhibits the property of liquid in dissolution due to the interaction between the solvent and the molecule of the solute, and the ability to separate the solute from the matrix is closely related to the density. Also, it shows the property of gas including high diffusibility relating to the permeability of the matrix, low surface tension, etc.

In practice, a material having a critical temperature and pressure for maintaining a supercritical state that is as low as possible is employed in fields for extracting and purifying the supercritical fluid. Carbon dioxide has a lower critical temperature and pressure than other materials used in making a supercritical state that is nontoxic to humans, environmentally friendly and inexpensive. Therefore, it is widely used as an extraction and purification solvent.

However, in spite of these advantages, carbon dioxide involves many problems in the extraction for various polar natural materials due to its low polarity. Accordingly, in an effort to solve these problems, the use of other supercritical fluids such as N₂O, the increase of temperature or pressure, and the addition of polar cosolvents, etc. can be typically mentioned. As for N₂O, the critical temperature and pressure are 36.5° C. and 70.6 bars, respectively. Accordingly, it shows a low critical point like carbon dioxide, and therefore, there are no great difficulties for making a supercritical fluid.

An increase in the temperature of a supercritical fluid causes a reduction in its density. However, it positively affects the diffusivity of the fluid, volatility of the desired component, and the desorption from the matrix.

Therefore, increasing the temperature and pressure as a control factor of a supercritical fluid has technological and economical limits, and it has little influence on the extraction efficiency of many compounds. Accordingly, as for supercritical carbon dioxide having low polarity problem, the method for adding a small amount of organic solvent has been used in order to enhance the polarity of the solvent.

Generally, water in the sample reduces the extraction efficiency of the supercritical fluid. However, in special cases, an increase in the water content in the sample causes the extraction efficiency to rise.

Since an alkaloid demonstrates various and potent physiological activities, its applicability to various compounds has been discussed since early on in the application of a supercritical fluid extraction technology for natural materials. However, it has a limit range of applicability that is relative to other natural materials.

A typical alkaloid-based component on which a supercritical fluid extraction technology has been applied is caffeine. Sugiyama et al (1985) estimated whether the temperature, pressure, and water in the sample, and the extraction time influenced the extraction efficiency in extracting caffeine from coffee fruit with a supercritical carbon dioxide. and optimal temperature and pressure were calculated (Sugiyama et al., J. Chromatogr., 332, 107-116 (1985)). Also, Sugiyama ascertained that the extraction efficiency increase according to the increase of water content in the sample is different from other compounds.

Elisabeth et al. (1991) reported that caffeine was separated and purified from coffee beans with a supercritical fluid chromatography (Elisabeth et al., Anal. Sci., 7, 427-431 (1991)). Ndiomu and Simpson (1988) also reported that if tetrahydrofuran or methanol is used as a cosolvent in extracting caffeine from a cola nut, the extraction efficiency increased up to about 2 times greater than that of pure carbon dioxide(D. P. Ndiomu, C. F. Simpson, Anal. Chim. Acta., 213, 237-243 (1988)). As for pyrrolizidine-based alkaloid, Schaeffer et al. (1989) disclosed that when monocrotaline was extracted from Crotalaria spectabilis with 5 to 10 mol % carbon dioxide (35 to 55° C., 10.34˜22.15 MPa), the extraction efficiency increased to 95% (Schaeffer et al., Ind. Eng. Chem. Res., 28, 1017-1020 (1989)). Also, as for pyrrolizidine alkaloid of Senecio species, Bicchi et al. (1991) found that carbon dioxide (55° C., 15 MPa), when 11% of methanol is added, produces a more effective yield and time of purification than the methanol soxhlet extraction method (Bicchi et al., J. Nat. Prod., 54, 941-945 (1991)).

As mentioned above, examples in which a supercritical fluid extraction technology had been applied to some alkaloid-based components has been reported. Yet, the applicable range was still limited relative to the other natural materials. The reasons why the range is limited are the high polarity of alkaloid components and the strong bond with plant matrixes in natural material. In order to solve these problems, research has been conducted to improve the extraction efficiency by increasing the amount of cosolvent added into carbon dioxide. However, owing to increases in the amount of the required cosolvent, the advantages that a supercritical fluid extraction technology have in comparison with the known organic solvent extraction method, i.e., non-toxicity to humans and environmentally friendly are compensated and therefore, it must try to find the solution. For example, although a supercritical carbon dioxide has been used for extracting thebaine, codeine and morphine, etc., it was found that approximately 25 to 50% of methanol as a cosolvent can use in order to compare with the prior organic solvent extraction method (see Janicot et al, J. Chromatogr., 505, 247-256 (1990)).

Given industry's increasing concern toward the environment, the technical task to be achieved by the present invention develops a method that can solve the problems of the prior organic solvent method, that is toxicity toward the environment and humans, non-selectivity for the desired component and high costs.

### SUMMARY OF THE INVENTION

The present invention provides a method of isolating at least one alkaloid from a plant. The method includes mixing plant material (e.g., leaves, stems, or roots) with carbon dioxide as a major solvent and 1 to 20 parts by weight of at least one alkaline cosolvent, wherein the cosolvent is selected from the group consisting of methanol, ethanol, water, and a mixture thereof, wherein 2 to 18 (v/v) % of diethylamine or triethylamine is dissolved in the alkaline cosolvent based on 100 parts by weight of carbon dioxide at a temperature of 70 to 90° C. and a pressure of 4,000 to 6,000 PSI in a supercritical extracting apparatus to extract the alkaloid; and isolating the alkaloid. The alkaloid can be isolated using chromatography. The temperature can be 75 to 85° C. and the pressure can be 4,700 to 5,300 PSI. The plant can be Scopolia japonica Nakai, and the alkaloid can be hyoscyamine and/or scopolamine. The plant can be Ephedra sinica Stapf., and the alkaloid can be selected from the group consisting of methylephedrin, norephedrin, ephedrin and pseudoephedrin. The plant can be Cephalotaxus wilsoniana Hayta, and the alkaloid can be cephalotaxin.

The invention also features purified hyoscyamine, scopolamine, methylephedrin, norephedrin, ephedrin, pseudoephedrin and cephalotaxin, produced by the methods of the invention.

### BRIEF DESCRIPTION OF THE FIGURES

FIG. 1 is a schematic view of a supercritical fluid extraction apparatus of the present invention.

FIG. 2 is a gas chromatogram obtained after a supercritical fluid extraction from Scopolia japonica Nakai was performed in accordance with the present invention. Reference numerals 1 and 2 indicate hyoscyamine and scopolamine, respectively.

FIG. 3 is a gas chromatogram obtained after a supercritical fluid extraction from Ephedra sinica Stapf. was performed in accordance with the present invention. Reference numeral 1 indicates methylephedrin, No. 2 indicates norephedrin, No. 3 indicates ephedrin and No. 4 indicates pseudoephedrin.

### DETAILED DESCRIPTION

Alkaloids exist not in the form of free base, but in the form of the salt thereof. Accordingly, many alkaloids exist in the vacuole of the plant cell and are basified by an acid solution. A salt of such alkaloids reduces the solubility towards non-polar solvents such as carbon dioxide and is the form that has been strongly bonded with the matrix of the natural materials.

Accordingly, the inventors grasped such characteristics of alkaloid-based components in natural materials and thereby converted alkaloids in the form of salt into free base form thereof, which is extractable with a non-polar solvent such as carbon dioxide and ascertained on the extraction efficiency of a supercritical fluid extraction technology.

Alkaloids selected as the desired component in the present invention and plants containing the same are as set forth below.

1. Hyoscyamine and scopolamine of Scopolia japonica Nakai. Scopolia japonica Nakai is a perennial herb belonging to the eggplant family. It is thick and strong at the root and trunk, has a height of 30 to 60 cm and a ramentum in the base thereof. The parts, which are used in a natural medicine, are roots, trunks of the roots and the leaves, and are used as a spasmolytic and an analgesic, and as materials for manufacturing scopolia extracts and scopolamine hydrobromide. It is reported that the components separated are hyoscyamine and scopolamine and apoatropine, and scopoletin and scopolin as a cumarin. Hyoscyamine of these shows strong parasympatholytic action such as a spasmolytic, a pupil and dilator action, anti-stress ulcer, motion of small intestine and secretoinhibitory. As for scopolamine, it has similar parasympatholytic action to hyoscyamine, but scopolamine shows stronger center action than hyoscyamine.

2. Methylephedrin, norephedrin, ephedrin and pseudoephedrin of Ephedra sinica Stapf. Ephedra sinica Stapf. belongs to the Ephedraceae family, a small herbaceous bush that naturally grows in Jilin in the northeastern provinces of China, grows on the high dry ground and hills, and grows to the height of 30 to 70 cm and a dioecism. The parts used in the natural medicine, are entire herbs; and it was reported that the components separated are methylephedrine, norephedrine, ephedrine and pseudoephedrin.

3. Cephalotaxin. Cephalotaxus wilsoniana Hayta is a tall evergreen tree of dioecism belonging to the Cephalotaxacea family. It naturally grows at 400 to 2,700 m above sea level. It reaches a height of up to 4 m, and there is no fuzz on the branches.

It was reported that the components contained in the Cephalotaxus wilsonina are alkaloids such as cephalotaxinon, acetylcephalotaxin, dimethyl cephalotaxin, epicephalotaxin, harringtonine, homoharringtonine, wilsonine, c-3epi-wilsonine, hydroxycephalotasine, isoharringtonine and the like (See, Chiu, et al., The illustrated medicinal plants of Taiwan, vol 3, p.32, SMC publishing, Taipei, Taiwan (1992)); and Powell et al., Phytochemistry, 11, 3317-3320 (1972)).

To identify whether the matrix affects the supercritical fluid extraction efficiency of a pure compound, 1 g of a filter cake disk (Advantec No. 2, Toyo Roshi Kaisha Tokyo Japan) was cut to the size of 1.0 cm and was then placed into an extraction bath. The solution of each of the subject compounds (0.2 mg/mL) was dropped in the extraction bath with a filter paper disk. It was then dried at 35 to 45° C. for 20 to 28 hours in a vacuum oven and immediately used.

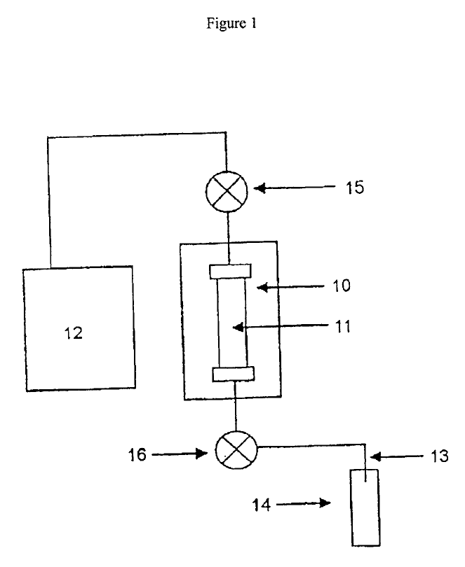

Isco supercritical fluid extractor, model SFX 3560, was connected with two Isco 260D syringe pumps (Lincoln Nebr., U.S.A.) and used as a supercritical fluid extraction machine. FIG. 1 illustrates a typical view for the supercritical fluid extraction machine used in the present invention.

Cooled carbon dioxide was injected first into the syringe pump (12) from a tank, and another syringe pump (12) was filled with a pre-selected cosolvent. Carbon dioxide and said cosolvent were blended with a T-mixer. The solvent, which had been preheated into the desired temperature by the preheater, was injected into the extraction bath (10:57 mm 20 mm, Isco) via the feed valve (15). The solvents injected were extracted after the progression of a constant static time to allow for sufficient contact with the solute.

After passage of said contact time, the vent valve (16) opened, and the extracts were entrapped in the collection vessel (14). The flowing rate was adjusted with the restrictor (13) that had been heated to a constant temperature of 80° C., and the extracts were collected into the collection vessel (14) filled with methanol to prevent depressurization.

After extraction, the extracts remaining in the restrictor (13) were collected into the collection valve (14) via a cleaning valve with carbon dioxide. The extracts that remained after the depressurization were again washed with methanol by the solvent pump connected with the restrictor (13).

The solubility of the extraction component in a supercritical fluid was measured. A sample absorbed on 1 g of a filter paper was extracted under supercritical fluid conditions so that optimal solubility could be achieved, in order to ascertain the influence that the matrix has on the extraction efficiency of the compound. The flow rate and temperature of the restrictor are 1.0 m L/min and 80° C., respectively.

As for extraction of a filter paper on which the alkaloids had been adsorbed, the filter papers were extracted as cosolvents with pure carbon dioxide; 1, 5 and 10% (v/v) of methanol, water and ethanol; and methanol, water and ethanol, wherein dimethylamine and trimethylamine and the like were dissolved.

One hundred (100) milligrams each of leaves, stem skins and roots of Scopolia japonica Nakai, Ephedra sinica Stapf. and Cephalotaxus wilsoniana Hayta and the like were placed into the extraction bath, and the supercritical fluid extraction was then conducted under the same conditions as those of a filter paper experiment. Fractions according to the extraction time were extracted in the fraction collector equipped with the supercritical fluid extraction apparatus for the given time, the vent valve was then closed and the extracts were again entrapped in the collection vessel.

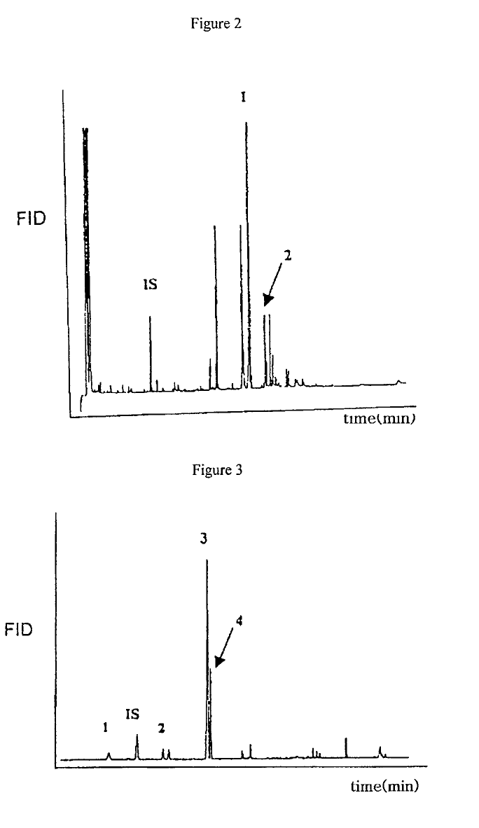

The final extracts of alkaloids of Scopolia japonica Nakai, in accordance with the present invention, were separated by gas chromatography, as shown in the data of FIG. 2. Also, the final extracts of Ephedra sinica Stapf. were separated by gas chromatography, as shown in the data of FIG. 3.

The present invention will be further described by the following examples, but should not be construed as being limited by them.

### EXAMPLE 1

### Extraction of Alkaloids, Hyoscyamine and Scopolamine from Scopolia japonica Nakai Using a Supercritical Fluid Extraction Technology

Solubility of pure supercritical carbon dioxide on hyoscyamine and scopolamine as a free base was measured as a basis experiment for the extraction efficiency of a supercritical carbon dioxide.

The salts of hyoscyamine and scopolamine did not dissolve at all in supercritical carbon dioxide under any temperature or pressure conditions (40˜60° C., 13.6˜34.0 MPa). Free base, on the other hand, showed a solubility of more than 510 μg/mL under all conditions.

It can be seen that the solubility of hyoscyamine and scopolamine was proportional to an increase of temperature and pressure under the experimental conditions; the solubility of the free base of hyoscyamine and scopolamine at 60° C., 34 MPa shows 5.9, 11 mg/mL, respectively, and are considerably dissolved in pure supercritical carbon dioxide. However, besides high solubility of free base of hyoscyamine and scopolamine in the pure supercritical carbon dioxide, no alkaloid was extracted from the plant under any temperature or pressure condition. It was known that Hyoscyamine and scopolamine exist in the vacuole of plant cell as water-soluble salt form, free base form of hyoscyamine and scopolamine likewise many alkaloids are well dissolved in a non-polar solvent such as a supercritical carbon dioxide, yet the salt form thereof does not hardly dissolve in the non-polar solvent.

In the next step, in order to increase the solubility of hyoscyamine and scopolamine salts in a supercritical carbon dioxide, polar solvents such as water and methanol were added.

Methanol and water were selected as cosolvents to improve the solubility of hyoscyamine and scopolamine salts toward a supercritical carbon dioxide. Methanol was most widely used in the extraction using a supercritical carbon dioxide because methanol can highly increase the polarity of carbon dioxide owing to high solvent polarity parameters, and also increases the swelling effect of a plant matrix when it is used in the extraction.

Upon observation of the change of the solubility according to the amounts of cosolvent added, it was found that methanol greatly increased the solubility of hyoscyamine and scopolamine salts. However, water did not greatly increase the solubility according to the amounts of cosolvent added. This is due to the fact that the level that was blended with carbon dioxide became smaller as the amount of water increased. The respective phases were observed through a view cell to ascertain the level of respective cosolvent in carbon dioxide.

On the other hand, based on the fact that hyoscyamine and scopolamine in the form of free base show considerable solubility under all experimental conditions, it tried to convert the salts thereof into a free base form. For this purpose, the cosolvents that were added in a supercritical carbon dioxide were basified. 1, 5 and 10% of cosolvent wherein 10% (v/v) diethylamine was mixed in methanol and water, respectively, were added to carbon dioxide and the solubility was measured.

By adding 1, 5 and 10% of the basic cosolvent, the solubility was greater than that of pure methanol or water. This is attributable to the conversion of hyoscyamine and scopolamine salts into free base thereof by the addition of methanol or water, which has been basified by diethylamine as a cosolvent.

From careful observation of the effect of cosolvent used in the present invention, it can be seen that the basic cosolvent shows a desorption of the desired alkaloid higher than methanol or water. In particular, the cosolvent with an addition of diethylamine into methanol is the most effective. When 10% of carbon dioxide is added, both the free bases and salts of hyoscyamine and scopolamine can be extracted up to about 50 to 80%.

In conclusion, as a result of measuring the effects of a cosolvent on the solubility of free bases and salts of hyoscyamine and scopolamine and on desorption from the matrix, it is understood that methanol with the addition of diethylamine did highly increase the extraction efficiency of a supercritical carbon dioxide.

The extraction efficiency of hyoscyamine and scopolamine from the root and the upper portion showed 95% and 85%, respectively, when 10% methanol with an addition of diethylamine was used, while the efficiency showed 10 to 54% for the existing extraction method using another cosolvent.

### EXAMPLE 2

### Extraction of Alkaloids, Methylephedrin, Norephedrin, Ephedrin and Pseudoephedrin from Ephedra sinica Stapf. Using a Supercritical Fluid Extraction Technology

This example measured the solubility in a supercritical carbon dioxide with an addition of a basic cosolvent on the assumption that ephedrin exists as a form of salt in a plant like hyoscyamine and scopolamine. Basic methanol with an addition of dimethylamine expectedly increased the solubility 2 times that of pure methanol. As for (−)-methylephedrin, methanol with an addition of diethylamine did not highly increase the solubility over pure methanol.

From the above, it can be seen that the conversion from salt to free base is not necessary because of the increased polarity of the solvent. Likewise, methanol with an addition of diethylamine, the cosolvent effect of water with an addition of diethylamine increased the solubility of salt of all ephedrin derivatives except for (−)-methylephedirn higher than water.

From the above, it can be ascertained that the use of a basic cosolvent is effective on the solubility of ephedrin derivative like hyoscyamine and scopolamine. Besides the solubility, in order to identify the level of desorption from matrix on which have been affected on the extraction efficiency, (−)-methylephedrin, (−)-norephedrin, (−)-ephedrin and (−)-pseudoephedrin hydrochloride, etc. were absorbed on the cellulose paper and the effect on the extraction efficiency was then measured. Likewise with solubility, it was therefore ascertained that basic cosolvents could increase the extraction efficiency of ephedrin derivatives higher than pure methanol or water.

It is found that basic cosolvent could highly increase desorption from the matrix as well as the solubility of the compound. Among the cosolvents used, when 10% methanol with the addition of dimethylamine is used, the extraction efficiency of all ephedrin derivatives was increased the greatest.

(−)-ephedrin and (+)-pseudoephedrin have diastereomers in terms of the structures thereof. It was found that ephedrin could be selectively extracted when a cosolvent with an addition of dimethylamine is used together with carbon dioxide, although it is not possible to selectively extract two materials with the known organic solvent extraction method.

### EXAMPLE 3

### Extraction of Alkaloid, Cephalotaxin from Cephalotaxus wilsoniana Hayta Using a Supercritical Fluid Extraction Technology

It is found that 0.0022% (22±0.51 μg/g) of cephalotaxin is contained in the leaves of C. wilsoniana from the result of experiment that 1 g of C. wilsoniana was quantified with the known organic solvent extraction method. When pure supercritical carbon dioxide was used under the condition of 40 to 80° C., 10.2 to 34.0 MPa, cephalotaxin was not extracted as expected. For the purpose of enhancing the yield, an extraction of less than 20% was shown under all conditions over the known organic solvent extraction as a result of extracting with the addition of 1, 5 and 10% volume ratio of methanol and water at 80° C. and 34.0 MPa.

However, when it was used with basic cosolvent, wherein 10% vol of diethylamine is added with methanol, the extraction resulted in 28% higher yield than the known organic solvent extraction method. As indicated in the extraction of a supercritical carbon dioxide for trophan-based component and benzylamine component, it was concluded that basic cosolvent caused the conversion of cephalotaxin salt in the plant to a free base thereof, which was well dissolved in a supercritical carbon dioxide.

Accordingly, it is considered that the method wherein basic methanol with the addition of 10% diethylamine was added to a supercritical carbon dioxide as a basic cosolvent of the present invention may be settled as a general method in the supercritical fluid extraction of basic alkaloids.

For the purpose of applying a supercritical fluid extraction technology as a new method for extracting and purifying alkaloid from a plant, the influence of various types of cosolvents on the yields and the selectivity of the desired components were measured. In conclusion, when 10 volume % of cosolvents such as methanol, ethanol and water, wherein 2 to 18 (v/v) % of diethylamine or triethylamine was dissolved were added to a supercritical carbon dioxide, it was found that the yields and the selectivity were to 1.5 and 4 times greater than the known organic solvent extraction method.

What is claimed is:

1. A method of isolating at least one alkaloid from a plant, said method comprising mixing plant material with carbon dioxide as a major solvent and 1 to 20 parts by weight of at least one alkaline cosolvent selected from the group consisting of methanol, ethanol, water, and a mixture thereof, wherein 2 to 18 (v/v) % of diethylamine or triethylamine is dissolved in said alkaline cosolvent based on 100 parts by weight of carbon dioxide at a temperature of 70 to 90° C. and a pressure of 4,000 to 6,000 PSI in a supercritical extracting apparatus to extract said at least one alkaloid; and isolating said at least one alkaloid.

2. The method of claim 1, wherein said at least one alkaloid is isolated using chromatography.

3. The method of claim 1, wherein said temperature is 75 to 85° C. and said pressure is 4,700 to 5,300 PSI.

4. The method of claim 1, wherein said plant is Scopolia japonica Nakai.

5. The method of claim 4, wherein said alkaloid is hyoscyamine.

6. The method of claim 4, wherein said alkaloid is scopolamine.

7. The method of claim 1, wherein said plant is Ephedra sinica Stapf.

8. The method of claim 7, wherein said alkaloid is selected from the group consisting of methylephedrin, norephedrin, ephedrin and pseudoephedrin.

9. The method of claim 1, wherein said plant is Cephalotaxus wilsoniana Hayta.

10. The method of claim 9, wherein said alkaloid is cephalotaxin.

11. Hyoscyamine, scopolamine, methylephedrin, norephedrin, ephedrin, pseudoephedrin, and cephalotaxin, produced by the method of claim 1.
